(19) United States
(12) Patent Application Publication
(10) Pub. No.: US 2008/0317860 A1
(43) Pub. Date: Dec. 25, 2008

(54) Use of a Polysaccharide Which is Excreted by the Vibrio Diabolicus Species For the Engineering of Non-Mineralized Connective Tissue

(75) Inventors: Karim Senni, Aulnay-Sous-Bois (FR); Corinne Sinquin, Nantes (FR); Sylvia Colliec-Jouault, Nantes (FR); Gaston-Jacques Godeau, Antony (FR); Jean Guezennec, Plouzane (FR)

Correspondence Address: MARSHALL, GERSTEIN & BORUN LLP, 233 S. WACKER DRIVE, SUITE 6300, SEARS TOWER, CHICAGO, IL, 60606, US

(73) Assignees: INSTITUT FRANCAIS DE RECHERCHE POUR L'EXPLOITATION DE LA MER (IFREMER), Issy Les Moulineaux (FR); UNIVERSITE RENE DESCARTES PARIS 5, Paris (FR)

(21) Appl. No.: 12/096,596

(22) PCT Filed: Dec. 6, 2006
(86) PCT No.: PCT/FR06/02668

§ 371 Date: Jun. 6, 2008

(30) Foreign Application Priority Data
Dec. 7, 2005 (FR) FR 05 12413

(51) Int. Cl. A61L 27/26; A61L 27/60; A61K 38/18; C12N 5/00

(52) U.S. Cl. 424/484; 514/12; 424/93.7; 435/395

(57) ABSTRACT

The invention relates to the use of a polysaccharide which is excreted by the Vibrio diabolicus species for the engineering of non-mineralised connective tissues, in particular for skin and cartilage engineering.

The present invention relates to the engineering of non-mineralized connective tissue, in particular covering tissue (skin, gum, cartilage, tendons).

Exopolysaccharide (EPS)-producing bacteria have been isolated from microorganisms originating from deep hydrothermal ecosystems. HE800 is an EPS produced by the Vibrio diabolicus strain. Its weight-average molecular mass is approximately 800 000 g/mol in the native state. It is characterized by an original linear repeating oside sequence consisting of 4 oside residues:

[(-3)-DGlcNacβ(1-4)DGlcAβ(-4)DGlcAβ(1-4)DGalNacα(1-)]n

HE800 has been described in the International application in the name of IFREMER published under number WO 98/38327 and also in the following articles: Raguénès et al., Int J Syst Bact, 1997, 47, 989-995 and Rougeaux et al., Carbohyd. Res., 1999, 322, 40-45. Many applications have been described for this exopolysaccharide. By way of example of an application, mention may be made of International application WO 02/02051, which describes the beneficial properties of HE800 in bone healing. No application for HE800 is known to date with regard to the engineering of non-mineralized connective tissue.

Connective tissue is characterized by the presence, between its cells, of a very abundant extracellular matrix.

The extracellular matrix constitutes the framework of non-mineralized connective tissue. It gives non-mineralized connective tissue its shape, its mechanical strength and its flexibility and performs important physiological functions. The organization of the collagen network is an essential element of tissue structuring. In fact, collagens, and in particular fibrillar collagens, constitute the predominant protein category in extracellular matrices, and in particular those of the gum dermis and of cartilage.

The extracellular matrix is also necessary for maintaining the differentiated state of the cells which synthesize and remodel it, in particular the mesenchymal cells (fibroblasts, myofibroblasts, chondrocytes, pericytes, etc.) which are the prize cells of non-mineralized connective tissue. Myofibroblasts, which proliferate during cicatrisation but persist during chronic inflammatory processes resulting in fibrosis setting in, are in particular distinguished.

The main objective of tissue engineering is the reconstruction both of human tissues and human organs. Several approaches are developed.

A first approach consists in implanting a guiding structure into a damaged tissue. The guiding structure serves as a mold for the tissue to be reconstructed. The structure may optionally be enriched with molecules for stimulating cell growth. By way of example of this tissue engineering approach, mention may be made of EP1555035, which describes a bioabsorbable implant consisting of a bridged collagen-glycosaminoglycan mixed matrix. This matrix constitutes a guiding structure whose objective is to pave the way for the regenerative potential of the tissue.

A second tissue engineering approach consists of the ex vivo reconstruction of tissue substitutes from living cells for in vivo or ex vivo uses. The objective is to reproduce the tissue architecture. The method, based on the production of a collagen gel, was the first to demonstrate the vast possibilities of tissue engineering. The first tissue reconstruction studies carried out demonstrated that the incorporation of fibroblasts into a collagen gel made it possible to produce dermal equivalents that can then be epidermalized by seeding keratinocytes at their surface. The final result, a living reconstructed skin, can achieve an excellent level of differentiation under appropriate culture conditions and many aspects of keratinocyte terminal differentiation can thus be reproduced. This method of tissue reconstruction has proved to be applicable for the development of many other organs for clinical purposes (transplantation) or fundamental purposes (in vitro tissue modeling). However, it is not definite that this method makes it possible to obtain tissues having the mechanical strength essential for their clinical application.

This mechanical aspect is one of the reasons which have prompted the development of a second method, based on the use of biomaterials that can be colonized by cells and their culture in vitro. Thus, once incorporated into these supports, the cells secrete varied amounts of extracellular matrix resulting in the reconstruction of a tissue structure close to the tissue of origin, but also comprising the biodegradable network of the biomaterial. By way of example of a tissue engineering method, mention may be made of WO 03/041568, which describes a three-dimensional matrix comprising a matrix of fibrin and fibroblasts. This matrix makes it possible to generate tissue equivalents which can be grafted.

One of the subjects of the present invention is a matrix with improved mechanical properties which promotes fibroblast proliferation.

The inventors have demonstrated, surprisingly and unexpectedly, that a polysaccharide having a weight-average molar mass of between 500 000 and 2 000 000 g/mol, characterized by a linear repeating oside sequence comprising the 4 oside residues:

[(-3)-DGlcNacβ(1-4)DGlcAβ(-4)DGlcAβ(1-4)DGalNacα(1-)]

has the following properties: it induces fibroblast strain selection, it stimulates fibroblast mobilization and proliferation in the extracellular matrix, it accelerates collagen fibrillation and thus promotes reconstruction of the extracellular matrix.

This polysaccharide makes it possible to reconstruct the collagen network of non-mineralized connective tissue, and it constitutes a support allowing the adhesion and cell proliferation of fibroblasts.

Thus, by virtue of its properties, the polysaccharide enables the production of fibrillar collagen matrix with improved properties. The collagen network of fibrillar collagen matrices comprising the polysaccharide exhibits better resistance against physical factors such as temperature and mechanical stresses. Finally, it promotes the culture of mesenchymal cells, in particular the culture of fibroblasts, and allows the preparation of tissue substitutes.

A subject of the invention is the use of a polysaccharide or of a salt of this polysaccharide having a weight-average molar mass of between 500 000 and 2 000 000 g/mol, preferably between 700 000 and 900 000 g/mol, characterized by a linear repeating oside sequence comprising the following 4 oside residues:

[(-3)-DGlcNacβ(1-4)DGlcAβ(1-4)DGlcAβ(1-4)DGalNacα(1-)]

for the purposes of engineering non-mineralized connective tissue.

A subject of the present invention is a collagen matrix comprising a polysaccharide or a salt of this polysaccharide having a weight-average molar mass of between 500 000 and 2 000 000 g/mol, preferably between 700 000 and 900 000 g/mol, characterized by a linear repeating oside sequence comprising the following 4 oside residues:

[(-3)-DGlcNacβ(1-4)DGlcAβ(1-4)DGlcAβ(1-4)DGalNacα(1-)]

Typically, the polysaccharide may be in the form of a salt.

Typically, the polysaccharide is a polysaccharide excreted by the Vibrio diabolicus species, having a size of between 500 000 and 2 000 000 daltons. Methods of preparation have been described in the following documents: WO 98/38327, Raguénès et al., Int J Syst Bact, 1997, 47, 989-995 and Rougeaux et al., Carbohyd. Res, 1999, 322, 40-45.

Typically, the collagen of the matrix is a collagen chosen from the group consisting of fibrillar collagens such as collagen type I, II, III, V and XI or of a mixture thereof. Preferably, the collagen is a collagen type I.

Typically, in order to produce such a collagen matrix, those skilled in the art will use the techniques commonly used for the production of collagen matrices from acid-soluble fibrillar collagens. In the presence of the polysaccharide according to the invention, acid-soluble fibrillar collagens naturally form fibrils after neutralization of the pH. Alternatively, the collagen matrix according to the invention may be obtained by bridging of the polysaccharide according to the invention with the collagen. In order to carry out the bridging, those skilled in the art will use the techniques commonly used for bridging polysaccharides with collagen. EP1374857 is an illustration of a bridging technique which can be used.

A subject of the present invention is also a matrix comprising the polysaccharide as described above, characterized in that the polysaccharide has been rendered insoluble by crosslinking using one or more crosslinking agents.

Typically, in order to crosslink the polysaccharide according to the invention so as to render it insoluble, those skilled in the art will use the techniques commonly used to crosslink polysaccharides. By way of examples of crosslinking agents, mention may be made of sodium trimetaphosphate, epichlorohydrin, divinylsulfone, glutaraldehyde and bisepoxyranes, for instance 1,4-butanediol bis(epoxypropyl)ether and 1,4-butanediol diglycidyl ether.

Advantageously, the matrices according to the invention may also comprise a growth factor which promotes colonization of the matrix by the mesenchymal cells, in particular by fibroblasts.

Preferably, the growth factor may be chosen from the group consisting of TGF-beta, PDGF, FGFs, BMPs (bone morphogenetic proteins), VEGF and CTGF (connective tissue growth factor).

Typically, the matrices according to the invention may serve as a resorbable or nonresorbable medical device or as an implant, or may be integrated into a medical device or into an implant. Such matrices will allow the mechanical and functional replacement of damaged structures with a minimum of adverse reactions. Once placed on the tissue or implanted into a damaged tissue, these matrices will serve as a guiding structure and will pave the way for the regenerative potential of the tissue. The presence of the polysaccharide within the matrix accelerates the regeneration by accelerating the restructuring of the connective tissue. It makes it possible to achieve complete regeneration such that the appearance of pathological situations of fibrotic or inflammatory type is prevented. The presence of the polysaccharide within the matrix also promotes ordered penetration after grafting of the matrix by the mesenchymal cells of non-mineralized connective tissue, such as the fibroblasts, while at the same time prompting these same cells to produce their own extracellular matrix.

By way of example, the medical device may be a dressing.

According to a preferred embodiment of the invention, the matrices according to the invention may also comprise mesenchymal cells which come from any non-mineralized connective tissue so as to constitute a connective tissue substitute, in particular a dermal, cartilage or tendon substitute. This substitute may be implanted in vivo. The matrix may comprise mesenchymal cells derived from the marrow or from circulating blood, fibroblasts or chondrocyte cells.

Advantageously, the mesenchymal cells which colonize the matrix will be dermal fibroblasts, so as to constitute a dermal substitute. The matrix may also comprise keratinocytes so as to constitute a skin substitute.

Advantageously, the mesenchymal cells which colonize the matrix will be chondrocytes so as to constitute a cartilage substitute.

According to another embodiment, the invention relates to a cell culture support, characterized in that the surface of the support on which the cells are cultured comprises the polysaccharide according to the invention.

Typically, the polysaccharide is in the form of a film, a membrane or a three-dimensional honeycombed structure, or a hydrogel.

According to another embodiment, the invention relates to a method of culturing mesenchymal cells, in particular fibroblasts, especially dermal fibroblasts, characterized in that said fibroblasts are cultured on a matrix according to the invention or on a support as described above.

The content of all the documents cited should be considered to be part of the present description.

The present invention will be illustrated more clearly hereinafter by means of the examples which follow. These examples are given only by way of illustration of the subject of the invention, of which they no way constitute a limitation.

### EXAMPLES

### 1 Materials and Methods

1.1. Preparation of the Exopolysaccharide HE800 from Cultures of Vibrio diabolicus (HE800 Strain)

Methods for preparing HE800 have been described in the following documents: WO 98/38327, Raguénès et al., Int J Syst Bact, 1997, 47, 989-995 and Rougeaux et al., Carbohyd. Res, 1999, 322, 40-45.

a) Cultures of Vibrio diabolicus

The HE800 strain is cultured on 2216E medium [Oppenheimer, J. Mar. Res. 11, 10-18, (1952)] enriched with glucose (30 g/l). The production is carried out at 30° C. and at pH 7.4 in a 2-liter fermenter containing 1 liter of the 2216E-glucose medium. After culturing for 48 hours, the must has a low viscosity (of the order of 40 centipoises at 60 rpm).

b) Purification of the Exopolysaccharide

The bacteria are separated from the must by centrifugation at 20 000 g for 2 hours, and the polysaccharide is then precipitated from the supernatant with pure ethanol, and several ethanol/water washes are then carried out with increasing proportions of ethanol, according to the method described by Talmont et al. [Food Hydrocolloids 5, 171-172 (1991)] or Vincent et al. [Appl. Environ. Microbiol., 60, 4134-4141 (1994)]. The polysaccharide obtained is dried at 30° C. and stored at ambient temperature. 2.5 g of purified polysaccharide per liter of culture were thus obtained.

1.2. Obtaining Fibroblasts

The experiments were carried out on fibroblasts of dermal origin and fibroblasts of gingival origin. These two types of mesenchymal cells adopt a very similar behavior with respect to HE800; consequently, the results obtained with the gingival fibroblasts can be extrapolated to the dermal fibroblasts.

1.2.1) Culture Media:

The cultures are carried out in a “complete” medium composed of Dulbecco MEM Glutamax I containing 100 U/ml of penicillin, 100 μg/ml of streptomycin and 2 μg/ml of fungizone (Gibco BRL Cergy Pontoise, France) supplemented or not supplemented (deficient medium) with fetal calf serum (FCS).

1.2.2) Origin of the Tissue Samples:

The dermal biopsies used are placed in culture within 3 hours of them being taken by the practitioner. The samples used are obtained after circumcision, from foreskins of clinically normal children. The gingival biopsies are taken from young patients (under 30 years old) with no pathological conditions. The biopsies are taken from gum attached to premolars extracted for orthodontic reasons. In addition, these gums are declared clinically normal by the practitioner. These biopsies are tissue remnants detached during the extraction and which have required no modification of the intervention.

1.2.3) Culturing:

The dermal and gingival samples are rinsed twice in a DMEM medium containing a higher than normal concentration of antibiotics (6× penicillin, 4× streptomycin, 2× fungizone) and then they are cut up into very small explants (≈2 mm²). These explants are placed, using a sterile Pasteur pipette or with the tip of a scalpel, in a 25 cm² culture flask, with the parenchymal side on the plastic. The dish is then stood up and left in this position for 15 minutes so that the explants adhere, dry, to the plastic.

The explants that have adhered are covered with a few drops of DMEM supplemented with 20% fetal calf serum (FCS). The culture dish is then placed in an incubator at 37° C. overnight, in an atmosphere composed of 5% CO₂ and 95% air. The following day, the supernatant is replaced with fresh medium containing 20% FCS; it is subsequently renewed every week. After three weeks, the fibroblasts have completely colonized the bottom of the dish (the keratinocytes present in the explant do not adhere under these culture conditions); subculturing is then carried out. The explants are removed using forceps, and the cells are rinsed twice with PBS and then trypsinized (trypsin-EDTA, Gibco). The trypsinization is then stopped by adding DMEM containing 10% FCS. The cells are counted on a counter (Coulter) and then reseeded into several culture dishes. They are, at this time, considered to be first passage and are maintained in a complete medium containing 10% FCS. When the cells are again confluent, another passage is carried out according to the same procedure, and this is continued up to the start of the experiments.

1.3. Preparation of Films and Culturing of Fibroblasts

1.3.1) Preparation of HE800 Films and Culturing of Fibroblasts

Surfacting is carried out by depositing 200 μl of a 2 mg/ml solution of HE800 at the bottom of the culture wells (24-well dish, 2 cm²). The culture dish is placed under a culture hood on a hotplate set to 37° C., for at least 5 hours. After evaporation, an HE800 film forms at the bottom of the dish. The gingival fibroblasts are seeded at a rate of 10 000 cells per well and cultured for 7 days. The cells are counted each day, some wells are fixed for the morphological study and the immunodetection of smooth muscle α-actin.

1.3.2) Preparation of Collagen Films and of Collagen-HE800 Composite Films and Culturing of Fibroblasts:

The collagen used is an acid-soluble collagen type I (2 mg/ml) obtained from rat tail (Institut Jacques Boy, Reims). Surfacting of the culture dishes is carried out by depositing 2001 of a mixture of collagen (40 μg in total) and HE800 (5, 50 or 200 μg in total).

The culture dish (24-well dish, or labtek, 2 cm² per well) is placed under a culture hood on a hotplate set to 37° C., for at least 5 hours. After evaporation, a film of collagen with or without HE800 forms at the bottom of the dish. Fibroblasts are seeded onto these films in order to be sure of the biocompatibility of the new culture surface.

1.3.3) Characterization of the Structure of the Films:

The collagen films and the composite films are fixed with absolute ethanol at −20° C. and then rehydrated so as to be stained with Sirius red (Junquera staining, collagen-specific). Thus, in Sirius red, all collagens are stained under transmitted light, but only correctly fibrillated collagens are capable of deviating polarized light.

1.4. Culturing in Lattices (Collagen Matrix): Preparation of Equivalent Non-Mineralized Connective Tissue

The lattices are made up with the same collagen I as that used to form the collagen films. After neutralization of the acid solution of collagen (3 mg/lattice), the gel containing the cells, and which is undergoing polymerization, is poured into a Petri dish 5 cm in diameter. HE800 is added to the collagen before the addition of the cells, at a rate of 150 μg, 300 μg or 600 μg per lattice (respectively 5%, 10% and 20% of the total amount of the collagen).

1.4.1) Preparation of the Stock Solution:

| Components | Amount | |
|---|---|---|
| DMEM (powder) | 5 | g |
| NaHCO₃ | 1.1 | g |
| Nonessential AA (100x) | 5 | ml |
| BiΔ H₂O (sterile) | 17 | ml |

Filter

| | | |
|---|---|---|
| BiΔ H₂O (sterile) | 250 | ml |

10 ml of fetal calf serum are added per 50 ml of stock solution.

1.4.2) Preparation of the Lattices:

All the steps for preparing the lattice are carried out in ice.

| | | |
|---|---|---|
| Stock solution (FCS) | 2.75 | ml |
| Collagen (2 mg/ml) | 1.5 | ml |
| NaOH (0.1 N) | 0.25 | ml |
| Cells (300 000/ml) | 0.5 | ml |

The lattice is shaken then poured into the Petri dish and then left for 5 min at 37° C.
After 1 h, the dishes are slightly shaken in order to detach the lattices from the edges.

The culture media are changed every week.

1.4.3) Characterization of the Lattices (Collagen Matrices):

At various culture times (11 and 40 days), the lattices are recovered, fixed in paraformaldehyde, and then prepared for paraffin embedding. Sections 7 μm thick are then cut on a microtome. Specific staining of these sections makes it possible to observe and study the structure and the cellularity of the reconstructed connective tissue. Some of the parameters demonstrated can subsequently be studied by image analysis and thus be quantified. The quality of the collagen fibrillation is observed after staining with Sirius red; the cellularity of the equivalent connective tissue could be estimated by an image analysis after staining the sections with hemalun-eosin.

1.4.4) Determination of the Number of Fibroblasts Contained in the Lattices:

Hemalun-eosin staining makes it possible to distinguish the cells from the matrix which surrounds them. This is because hemalun stains the cell nuclei blue-black, whereas eosin stains the cytoplasms and the extracellular structures (eosinophilic) more or less intensely red. The contrast thus created makes it possible to distinguish each cell under a microscope equipped with a CDD camera connected to a semi-automatic image analyzer. The cells which are in the fields defined by the microscope magnification are then counted in the lattices at 11 and 40 days. About ten fields per section were analyzed. Two groups of cells can thus be differentiated according to their geographical situation: firstly, the cells which are inside the lattice (collagen matrix) and, secondly, the cells which are at the periphery of the lattice. In order to calculate the cellularity of the equivalent connective tissue, each lattice is considered to be cylindrical, the periphery of the lattice being defined as a crown 10 μm thick (equivalent to the diameter of two cell strata) representing 2% of the total volume of the lattice.

1.5. Indirect Immunodetection of Smooth Muscle α-Actin

The fixed cells are repermeabilized in 70% ethanol (20 min) and then rehydrated in PBS (10 min). The endogenous peroxidases are blocked with a methanol (30%), H₂O₂ (0.3%) solution. This operation is followed by rinsing with PBS (2 min), and then by blocking of the nonspecific antigenic sites with a PBS/1% skimmed milk solution (1 h). The cultures are then incubated with a primary antibody (mouse IgG) directed against human α-actin (1/30; 50 min) and then rinsed with PBS (3×10 min). The cells are then incubated in the dark for 60 min with a biotinylated anti-mouse IgG antibody (1/200), rinsed with PBS (3×10 min), and then incubated with peroxidase-coupled streptavidin (1/200).

After rinsing (PBS 3×10 min), the peroxidase activity is revealed with 3,3′-diaminobenzidine in a Tris/HCl buffer (100 mM, pH 7.2-7.4) containing 0.1% of H₂O₂ (15 min, in the dark). The peroxidase activity causes a brown fibrillar material to appear (corresponding to the α-actin microfilaments) in the cytoplasm of the positive cells.

The products used come from the company Dako. The controlled experiments concerning the immunodetection of smooth muscle α-actin were carried out by omitting the primary antibody and/or by using a secondary antibody of an animal species other than that which made it possible to obtain the primary antibody.

### 2. Results and Discussion

2.1. Proliferation of Gingival Fibroblasts Cultured on HE800 Film

The culture surfaces were treated with HE800 in order to form a polysaccharide film at the bottom of the dishes. During the first days of culture (days 2 and 4), it is observed that the number of cells seeded in the HE800-coated dishes is much lower than the number of those in the control dishes (Table I). On the other hand, on the last day of the experiment, an inversion of these results is noted (Tables I and II). The curves presented show that the cells cultured on HE800 film observe a lag phase, before entering into the exponential growth phase, which is longer than that expressed by the cells cultured on plastic. Furthermore, although the number of cells in the control cultures reach a plateau at the latest days of the experiment (cf. Table III), the cultures on HE800 film continue to proliferate. The observations made during culturing or after fixing of the cells make it possible to put forward hypotheses as to the cell behaviors expressed under the various culture conditions.

The cultures on HE800 film are characterized, in the first days of culture, by the presence of numerous cells which do not adhere to the support. This nonadhesion may explain the delay in proliferation observed in the cell counts in these cultures.

The control cells are distributed uniformly in the dish, without any particular orientation, whereas the cells seeded on HE800 film become organized in strings at the center of the dish. These results show the effect of HE800 on the cell adhesion. In fact, the cell groupings which are normally observed, in gingival cultures, have no specific orientation. After the first 2 days of culture, these strings of cells begin to form a circular central structure, becoming denser exclusively toward the center (centripedal proliferation). Many cells can also be observed at the periphery of the dish, but with no particular orientation. Some cells may be present in the areas separating the cell groupings, they are isolated and appear to be much more drawn out in length than the other cells of the HE800 or even control dishes.

The immunocytochemical labeling regarding the smooth muscle α-actin shows:

in the controls, many positive cells are next to cells not expressing these microfilaments.

in the cultures in the presence of HE800, the cells present in the central circular formations do not express smooth muscle α-actin; on the other hand, cells expressing this actin isoform can be found at the periphery of the dish.

These results reflect a selection of fibroblast strains; in fact, some cells may not naturally express the membrane receptors required for them to adhere to the HE800 film. Among the nonadherent fibroblast subpopulations are those which express smooth muscle α-actin, i.e. myofibroblasts. In the control experiments (emission of the primary antibody or use of an inappropriate secondary antibody), no positive was observed.

TABLE I
Variation in the number of cells per well during the culture

| | Day 2 | Day 4 | Day 7 |
|---|---|---|---|
| Control | 14 812 | 45 610 | 52 980 |
| HE800 | 10 035 | 36 995 | 64 247 |

TABLE II
Proliferation percentages

| | Day 2 | Day 4 | Day 7 |
|---|---|---|---|
| Control | 100 | 100 | 100 |
| HE800 | 67.75 | 81.11 | 127.27 |

TABLE III
Doubling time (hours)

| | Dt 0-2 | Dt 2-4 | Dt 4-7 |
|---|---|---|---|
| Control | 84.68 | 29.58 | 333.2 |
| HE800 | 9522.64 | 25.50 | 90 |

2.2. Structuring of Collagen Type I in the Presence of HE800

2.2.1) First Observations

In order to prepare the films comprising both collagen and exopolysaccharide HE800, solutions of HE800 and collagen I are premixed before deposition onto the culture dishes. Surprisingly, it was noted that the addition of the bacterial exopolysaccharide to the collagen solution caused the appearance of a dense, white-colored agglomerate. This agglomerate could be spread on a histological slide and then stained with Sirius red, a collagen-specific dye. These histological slides show that the material spread on the slide is effectively stained with Sirius red. Furthermore, the observation of a material causing polarized light to deviate in various directions clearly shows the presence of collagen in its fibrillar form.

2.2.2) Organization of Collagen/HE800 Composite Films:

The various films deposited are composed of:
(1) collagen (40 μg)
(2) collagen (40 μg)+HE800 (50 μg)
(3) collagen (40 μg)+HE800 (200 μg).

Observation of the bottom of the dishes under a microscope shows, for the films (3), the appearance of a dense network composed of long filaments. The films (2) comprise some much shorter fibers, whereas the films (1) comprise virtually none. The 3 films stain with Sirius red, but only the film (3) shows a fibrillar network which causes polarized light to deviate.

These results indicate that HE800 promotes the formation of collagen fibers, but also allows better resistance of the collagen network against physical factors such as temperature and mechanical stresses.

2.3. Non-Mineralized Connective Tissue: Photon and Electron Microscopy

The cells are cultured in a collagen matrix (three-dimensional culture model) in order to mimic as closely as possible the cell/matrix interactions observed in connective tissue. These lattices or equivalent connective tissues are composed of collagen I alone (controls) or of collagen I and HE800 in various proportions (amount of EPS=20, 10 and 5% relative to the amount of collagen contained in the lattice, i.e. 300, 150 and 75 μg, respectively).

2.3.1) Retraction of the Lattices:

The first parameter studied is the rate of retraction of the lattices: the retraction curves for the control lattices and for the lattices comprising HE800 are similar. Despite these similarities, it is noted that the HE800 lattices have a slower retraction rate than the control lattices during the early days of culture. After the 11^{th} day, the retraction of the lattices is almost complete.

2.3.2) Number of Fibroblasts Contained in the Lattices:

The number of cells present in each lattice varies, at the two culture times, between 180 000 and 250 000 cells. The number of cells at the periphery represents 2 to 12% of the total number of cells. These data are compatible with what has been described in the literature for this culture model. The results in Tables IV, V and VI show the number of cells per unit of volume (mm³) present in the entire lattice and in its various regions.

The total volumetric cell densities of the lattices after 11 and 40 days are between 3200 and 5900 cells/mm³ (cf. Table IV). These values are comparable to those found in a normal human connective tissue, as has been previously described (Miller et al., Exp Dermatol. 2003 August; 12(4): 403-11). The physiological cellularity of the control lattices and of the lattices comprising HE800 therefore attests to the validity of the culture model used and to the compatibility of HE800 with this physiological model.

The total cell density (cf. Table IV) of the control lattices does not vary whatever the culture time. At the 11^{th} day of culture, the total cell density of the HE800 lattices is 25 to 40% lower than those of the control lattices. At the 40^{th} day of culture, the cell densities of the control lattices and of the HE800 lattices are equivalent. The variations in the cell densities observed inside the lattices (cf. Table V) reproduce exactly those of the entire lattice. The topological organization of the cells of the peripheral crown (Table VI) on the other hand diverge completely from those of Tables IV and V:
at 11 days of culture, the cell densities of the peripheral crown are 4 times higher than those of the interior of the lattice and show only slight variation between the control equivalent connective tissues and the equivalent connective tissues comprising HE800 (Table VI).
at 40 days of culture, a large decrease in the peripheral cellularity is observed. While this decrease is only 40% for the control lattices, it reaches 100 to 250% for the lattices containing HE800. The cell density of the control lattices remains 3 times higher at the periphery than at the interior (Table IV and VI); on the other hand, these densities are comparable for the HE800 lattices.

The overall cellularity of the HE800 lattices is lower than that of the control lattices early on in the culture, and then becomes equivalent later on in the culture. These variations, which have an effect on the cell densities of the internal regions, can be explained by a stimulation of cell proliferation, or massive migration of peripheral cells to the interior.

In fact, at the periphery of the lattices, the number of cells decreases over the culture time; this decrease is particularly accentuated in the lattices comprising HE800 (decrease by 2 to 3.5 times of the number of cells). This decrease can be explained by a loss of adhesion of the peripheral cells, which detach from the extracellular matrix, and/or a massive migration of these cells to the interior. This explains the overall gains in cellularity, over time, in the lattices containing HE800.

TABLE IV
Cell density in the entire lattice: number of cells per
mm³ (between parentheses: lattice diameter in mm)

| Culture | | | | Variation between |
|---|---|---|---|---|
| time | 11 days | | 40 days | 11 and 40 days |
| Controls | 5924 | (8.3) | 5695 (8) | −4% |
| HE800 20% | 4468 | (8) | 6150 (8) | +27% |
| HE800 10% | 3899 | (9.7) | 6000 (8) | +35% |
| HE800 5% | 3491 | (1.03) | 5023 (8) | +30% |

TABLE V
Cell density inside the lattice: number of cells per mm³

| Culture | | | Variation between |
|---|---|---|---|
| time | 11 days | 40 days | 11 and 40 days |
| Controls | 5568 | 5520 | −3% |
| HE800 20% | 4153 | 6085 | +22% |
| HE800 10% | 3517 | 5938 | +41% |
| HE800 5% | 3220 | 5005 | +36% |

TABLE VI
Cell density at the periphery of the
lattice: number of cells per mm³

| Culture | | | Variation between |
|---|---|---|---|
| time | 11 days | 40 days | 11 and 40 days |
| Controls | 20 134 | 14 382 | −40% |
| HE800 20% | 23 367 | 6531 | −258% |
| HE800 10% | 22 161 | 9979 | −122% |
| HE800 5% | 17 731 | 5939 | −199% |

Conclusion: HE800 promotes the proliferation of dermal fibroblasts in the extracellular matrix and/or promotes their mobilization, i.e. the selection, migration and massive penetration of the peripheral cells.

2.4.3) State of the Collagen Matrix:

2.4.3.1) Photon Microscopy

Sirius-red staining (Junquera staining) makes it possible to specifically stain collagens; in the skin, for example, its collagens appear in the form of a red-colored, loose filamentous structure.

The Sirius-red stainings of the histological sections after observation under transmitted light and polarized light show that the addition of HE800 during the formation of the lattice allows the formation of a matrix which is much more dense and after much shorter periods of time than in the control lattices.

For example, the density of the control collagen matrix after 40 days of culture is equivalent to that observed in the collagen matrices formed in the presence of HE800 at 11 days of culture. This effect on the density is much greater at the lowest doses (10%, 5%).

2.4.3.2) Electron Microscopy

Electron microscopy was carried out on equivalent connective tissues cultured for 11 days. The cells were seen to have a good ultrastructural state, whether in the controls or in the lattices formed in the presence of the various concentrations of HE800.

No collagen fibers could be observed in the control lattices; the lattices comprising 20% of HE800 make it possible to observe some fibrillar elements held in a gel consisting of the exopolysaccharide. The lattices comprising 10% and 5% of exopolysaccharides are very different; specifically, numerous collagen fibers are present, they are distributed throughout the lattice and some exhibit a periodic striation. These collagen fibers or fibrils are trapped in the gel consisting of the HE800.

Conclusion: The HE800 accelerates collagen fibrillation and promotes the constitution of an extracellular matrix.

1. A collagen matrix comprising a polysaccharide or a salt thereof having a weight-average molar weight of between 500,000 and 2,000,000 g/mol, characterized by a linear repeating oside sequence comprising the following 4 oside residues:
[(-3)-DGlcNacβ(1-4)DGlcAβ(1-4)DGlcAβ(1-4)DGalNacα(1-)]

2. The matrix as claimed in claim 1, wherein said polysaccharide is a polysaccharide excreted by the Vibrio diabolicus species.

3. The collagen matrix as claimed in claim 1, wherein the collagen is a fibrillar collagen chosen from the group consisting of collagen type I, II, III, V and XI and mixtures thereof.

4. The matrix as claimed in claim 3, wherein the collagen is a collagen type I.

5. A matrix comprising the polysaccharide as defined in claim 1, wherein the polysaccharide has been rendered insoluble by crosslinking using one or more crosslinking agents.

6. The matrix as claimed in claim 1, further comprising one or more growth factors which promote colonization of the matrix by mesenchymal cells.

7. The matrix as claimed in claim 6, wherein the growth factor is chosen from the group consisting of TGF-beta, PDGF, FGF, VEGF, BMPs and CTGF.

8. The matrix as claimed in claim 1, further comprising mesenchymal cells.

9. The matrix as claimed in claim 8, wherein the mesenchymal cells are cells derived from the marrow or from circulating blood, fibroblasts or cartilage cells.

10. The matrix as claimed in claim 9, wherein the mesenchymal cells are dermal fibroblasts.

11. The matrix as claimed in claim 10, further comprising keratinocytes.

12. The matrix as claimed in claim 8 wherein the mesenchymal cells are chondrocytes.

13. A non-mineralized connective tissue substitute comprising the matrix as claimed in claim 8.

14. The substitute as claimed in claim 13, comprising a tendon substitute.

15. A dermal substitute comprising the matrix as claimed in claim 10.

16. A skin substitute comprising the matrix as claimed in claim 11.

17. A cartilage substitute comprising the matrix as claimed in claim 12.

18. A medical device or implant comprising the matrix as claimed in claim 1.

19. The medical device as claimed in claim 18, wherein the medical device is a dressing.

20. A method for the in vitro culture of mesenchymal cells, comprising culturing the mesenchymal cells on the matrix as claimed in claim 1.

21. (canceled)

22. The matrix of claim 1 wherein the polysaccharide or salt has a weight-average molecular weight between 700,000 and 900,000 g/mol.

23. The matrix of claim 6 wherein the growth factor promotes colonization of the matrix by fibroblasts.
